(19) United States
(12) Patent Application Publication
(10) Pub. No.: US 2008/0119699 A1
(43) Pub. Date: May 22, 2008

(54) Method for detecting at least one substance in a metabolic process of a living being and device for carrying out the method

(76) Inventors: Klaus Abraham-Fuchs, Erlangen (DE); Ralph Gareus, Forchheim (DE); Arne Hengerer, Erlangen (DE); Heiki Sieber, Bamberg (DE)

Correspondence Address: HARNESS, DICKEY & PIERCE, P.L.C., P.O.BOX 8910, RESTON, VA, 20195, US

(21) Appl. No.: 12/010,246

(22) Filed: Jan. 23, 2008

Related U.S. Application Data
(62) Division of application No. 10/855,406, filed on May 28, 2004.

(30) Foreign Application Priority Data
May 30, 2003 (DE) 10324600.2

(51) Int. Cl. A61B 5/00

(52) U.S. Cl. 600/317

(57) ABSTRACT

A method is for detecting at least one substance in a metabolic process of a living being. A molecular contrast agent is administered to the living being. The method is able to highlight the substance being detected continuously over time at least one location of the living being. A device may be used for carrying out the method.

This application is a divisional application of, and claims the benefit of priority under 35 U.S.C. §120 from, pending application Ser. No. 10/855,406 filed May 28, 2004 which claims priority under 35 U.S.C. §119 on German patent application number DE 103 24 600.2 filed May 30, 2003, the entire contents of each reference are hereby incorporated herein by reference.

### FIELD OF THE INVENTION

The invention generally relates to a method for detecting at least one substance in a metabolic process of a living being and a device for carrying out the method.

### BACKGROUND OF THE INVENTION

There are known devices for measuring the variation over time of the concentration of a metabolic parameter, in particular portable sensor devices. These can include, for example, blood glucose measuring devices or devices for measuring the oxygen saturation of the hemoglobin. Specific glucose or hemoglobin dioxide sensors measure the target substance directly.

For example, DE 195 40 456 A1 also discloses an artificial pancreas, including a glucose sensor based on evaluation of a physical parameter of blood, an insulin reservoir, an insulin dispensing device and a microprocessor as a controller. The glucose sensor determines the glucose concentration in the blood virtually continuously and causes the insulin dispensing device to dispense insulin from the insulin reservoir according to requirements, i.e. in dependence on the glucose concentration.

In this case, the artificial pancreas can be fully implanted or can be arranged externally on the patient, the glucose sensor lying for example in a vessel of the patient, while the insulin dispensing takes place subcutaneously. The glucose sensor thereby determines the blood glucose concentration and its dynamics, that is to say the variation over time, virtually continuously and passes this information on to the controller.

### SUMMARY OF THE INVENTION

An object of an embodiment of the invention is to provide an improved method for detecting at least one substance in a metabolic process of a living being and an associated device with which it is possible inter alia also to detect a difficulty detectable substance in a living being.

To monitor the variation over time of a metabolic process, a molecular, in particular biomolecular, contrast agent is administered. For example, it is injected into the body of a living being, taken orally, etc. The agent bonds specifically to a metabolic component, for example a transforming molecule, in particular an enzyme, or a metabolic product, for example a substrate, of the metabolic chain of interest. Or it is activated into a signaling state by way of a biochemical reaction with this metabolic component or metabolic product. As a result, metabolic components or metabolic products which it is not possible to detect directly, in particular through a skin of the living being, at all, or only with great effort, are detectable. Thus, the rate of a metabolic process may be calculated based on results of the detection wherein an unknown (or indirectly detectable) parameter may be determined based on a known (or directly detectable) parameter.

In this case, the specifically bonded molecule or the activated contrast agent can be traced by use of a detector. The detector may be integrated in a corresponding device which can be carried over a relatively long period of time on or in the body of the living being. The device optionally has a device/method for signal evaluation and/or signal transmission.

In this respect, there are numerous known contrast agents which, by way of molecular engineering, bond highly specifically to target molecules, for example proteins. Subsequently, by use of a signaling substance coupled to them, they can emit a signal which can be measured outside the body. As such, the presence of the target molecules, and if appropriate their concentration, can be detected.

Examples of such contrast agents are described in U.S. Pat. No. 6,083,486. These include a number of functional components, the alternatives of which are listed in column 1, line 61 to column 3, line 29 of U.S. Pat. No. 6,083,486.

According to the prior art, these biomolecular contrast agents, in particular fluorescent contrast agents, are used for optical imaging and in pharmaceutical research with small animals, for example mice. This primarily involves presenting the spatial distribution of a target molecule in the body, which serves for example for the diagnosis and localization of a tumor. Since, however, such contrast agents can be used for example to detect proteins, DNA and/or RNA segments, which are the starting point or reagent of a metabolic process chain, such contrast agents are also suitable for highly specifically characterizing a metabolic process of interest.

Biomolecular contrast agents such as are described in the aforementioned U.S. Pat. No. 6,083,486, may also be designed in such a way that a distinction can be made between active and inactive process variants or that the activity of enzymes themselves or of decomposition products produced by the enzymes can be measured. According to an embodiment of the invention, the transformation of a reagent product or a decomposition product that is characteristic of a biological metabolic process can thereby be measured in a highly specific way, and consequently the metabolic rate can be concluded.

In this case, the concentration of a molecule from a metabolic chain can be measured over a relatively long period of time. This allows answers to be found to the medically motivated questions of whether a specific metabolic process, for example a metabolic process which is characteristic of a tumor, or a decomposition process, is taking place at all for example in the liver. It further allows one to determine at what time intervals, expressed in minutes, hours or days, the metabolic process is taking place; with which intensity the metabolic process is taking place; and/or whether the intensity is currently increasing, decreasing or remaining constant.

For detecting the contrast agent, it may in this case be assumed that the location at which the concentration of the metabolic marker of interest is present in a meaningful concentration is known. For example, many metabolic markers are transported in a detectable concentration in the bloodstream and are consequently measurable at any point of the cardiovascular system. Other suitable measuring locations may be on the surface of the body directly over organs of the body, for example the liver, the kidneys or lymph nodes, or directly over lesions, for example tumors and wounds from operations, etc.

A portable device which can be carried on the body and contains a detector which can measure the signals emitted by the molecular contrast agent over the course of time, for example as a fluorescence signal, magnetic field or radioactive radiation, and can record them over time, is placed at this location identified as suitable. For example with respect to the contrast agents of U.S. Pat. No. 6,083,486, the wavelengths of which are specified in Table 1 of U.S. Pat. No. 6,083,486, the detector must be suitable for detecting the wavelengths emitted by the fluorescent contrast agent. In this case, selective detection is carried out in an advantageous way using an upstream optical filter.

### BRIEF DESCRIPTION OF THE DRAWINGS

The present invention will become more fully understood from the detailed description of preferred exemplary embodiments given hereinbelow and the accompanying drawing, which is given by way of illustration only and thus are not limitative of the present invention, and wherein:

FIG. 1 shows a device for continuously detecting at least one substance highlighted by a molecular contrast agent in a metabolic process of a living being,

FIG. 2 shows embodiments of the device as an arm band and implant and

FIG. 3 shows embodiments of the device as an auditory canal device and ear clip.

### DETAILED DESCRIPTION OF THE PREFERRED EMBODIMENTS

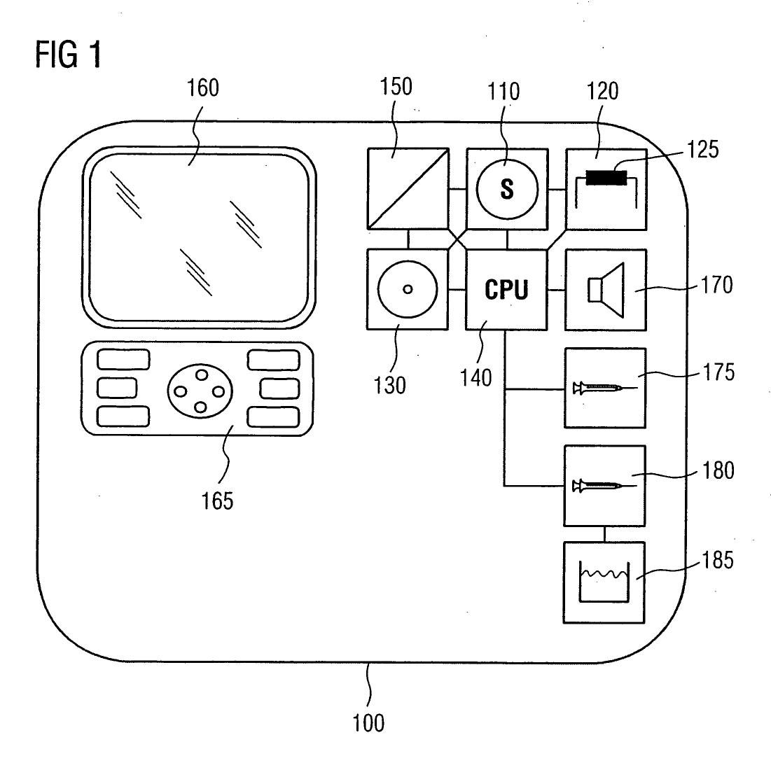

FIG. 1 shows as an exemplary embodiment of the invention a device 100 for continuously detecting at least one substance in a metabolic process of a living being, which is highlighted by a molecular contrast agent, with a detector unit 110 for detecting the molecular contrast agent administered to the living being. In one embodiment of the invention, a fluorescent contrast agent is used, and the detector unit 110 for this contrast agent that is integrated in the device 100 is accordingly formed as a detector for measuring fluorescence signals, comprising a CCD chip for example.

In a further embodiment of the invention, the contrast agent is designed such that it can be enriched in the spatial vicinity of the detector unit 110 by way of an enriching unit 120 that is integrated in the device 100. This can be achieved for example by the contrast agent having magnetic properties, for example including iron oxide particles, and the enriching unit 120 including a magnet, so that the magnetic molecules of the contrast agent are drawn toward the magnet or, when they are transported through the system of vessels, partly immobilized in the vicinity of the magnet in the blood vessel. The magnet may be designed as an electromagnet 125, so that the immobilization of the molecules can be switched on and off. By such a procedure, the molecules can be enriched in the vicinity of the detector unit 110 and consequently an intensified measurement signal can be obtained.

The device 100 also include a memory unit 130, in which the variation over time of the measurement signal recorded by the detector unit 110 is stored. Furthermore, at least one interface unit 150 is provided, for the wireless and/or cable-bound transmission of the signals to a receiver for the evaluation and/or presentation of the signals. In one embodiment, the measurement signal can also be transmitted to a receiver without intermediate storage, by way of wireless transmission.

The device 100 also includes a display unit 160 for the alphanumeric and/or graphic display of the intensity of the metabolic process and/or for indicating the trend of the metabolic process in the sense of increasing, decreasing or remaining constant. It further includes an alarm transmitter unit 170, which becomes active if the signal goes above or below a preset threshold value of the measurement signal. Finally, the device 100 also includes an input unit 165, for example a keypad, for setting device options or entering data, for example a threshold value; and a processor unit 140 for evaluating the measurement data and/or a dosing unit 175 for medicaments.

In one embodiment, at least the detector unit 110 and/or the processor unit 140 are in this case set up in such a way that only the presence or absence of the measurement signal or of the measurement signal lying above the threshold value of a background activity or a background noise is detected and used to conclude the presence or absence of the metabolic process being observed. In another embodiment, in which it is assumed that the intensity of the measured signal is proportional to the concentration of the component of the contrast agent that highlights the target molecules, consequently proportional to the concentration of the target molecule and to the intensity of the metabolic process, the detector unit 110 in particular is set up for intensity-sensitive detection.

Furthermore, in one embodiment the device 100 is set up in such a way that a point in time at which the contrast agent is delivered can be entered into the device 100 and stored, in order to be able to take this point in time into account in the evaluation and, if appropriate, presentation of the signals. Similarly, in one embodiment the amount of the contrast agent administered can be entered.

Furthermore, the device 100 includes a reservoir unit 185 for the contrast agent and an injection unit 180 for the contrast agent, by which parts of the contrast agent located in the reservoir unit 185 can be injected into the living being in a dosed amount on command or at time intervals which can be preset.

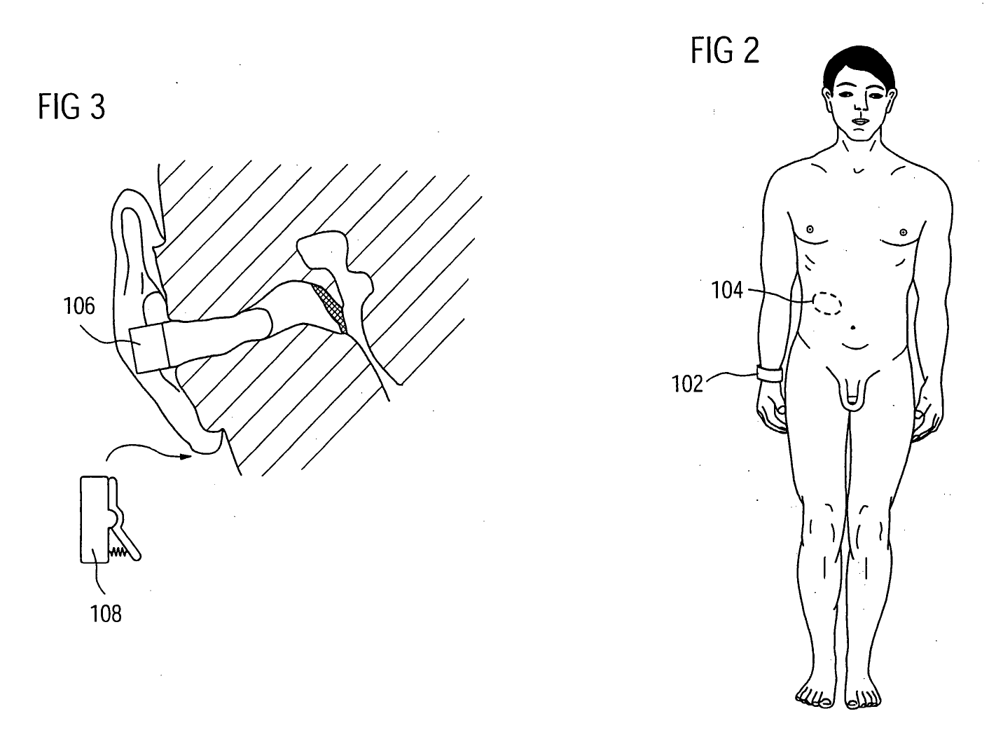

FIG. 2 shows as exemplary embodiments of the invention configurations of the device 100 as devices which can be carried on the body of a patient in the form of an arm band 102 and/or as an implant 104. In the case of the arm band 102, for example, the detector unit 110 is integrated into the arm band 102 in such a way that the detector unit 110 is placed in a suitable way over a main blood vessel of the wrist.

Finally, FIG. 3 shows as exemplary embodiments of the invention a sectional diagram in the region of the left-hand auditory canal of the patient from FIG. 2 with configurations of the device 100 as an auditory canal device 106 and an ear clip 108. In this case, the auditory canal device 106 is designed in such a way that signals from the system of vessels of the auditory canal can be picked up by the detector unit 110. In the case of the ear clip 108, the detector unit 110 is integrated in such a way that the detector unit 110 is placed in a suitable way over the system of vessels of the ear lobe.

In embodiments of the invention, the configurations of the devices 102 to 108 which can be carried on the body do not comprise all the units 110 to 185 described with respect to FIG. 1, and/or at least two of the units 110 to 185 are arranged such that they are distributed among at least two of the configurations of the device 102 to 108. For example, the detector unit 110 may be accommodated in the ear clip 108 and the display and input units 160 and 165 may be accommodated in the arm band 102.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1-20. (canceled)

21. A device for detecting at least one substance in a metabolic process of a living being, comprising:
means for detecting a molecular contrast agent, administered to the living and able to highlight the at least one substance, continuously over time at least one location of the living being; and
means for detecting a component of the molecular contrast agent that highlights the at least one substance.

22. The device as claimed in claim 21, wherein the device is carriable on the body of the living being.

23. The device as claimed in claim 21, wherein at least part of the device is integrated in an arm band.

24. The device as claimed in claim 21, wherein at least part of the device is integrated in an ear clip.

25. The device as claimed in claim 21, wherein at least part of the device is implantable in the body of the living being.

26. The device as claimed in claim 21, wherein at least part of the device is carriable in an auditory canal of the living being.

27. The device as claimed in claim 21, further comprising means for optically detecting a component of the contrast agent that highlights the at least one substance.

28. The device as claimed in claim 27, wherein the means includes a CCD chip.

29. The device as claimed in claim 21, further comprising at least one of an alphanumeric and graphic display unit.

30. The device as claimed in claim 21, further comprising an alarm transmitter unit.

31. The device as claimed in claim 21, further comprising an input unit for entering data.

32. The device as claimed in claim 21, further comprising a dosing unit for at least one medicament.

33. The device as claimed in claim 21, further comprising an injection unit for the contrast agent.

34. The device as claimed in claim 21, further comprising an interface unit for at least one of wireless and cable-bound transmission of data.

35. The device as claimed in claim 21, further comprising means for enriching the contrast agent.

36. The device as claimed in claim 35, wherein the means for enriching includes, in a case of a contrast agent which can be magnetically enriched, a magnet formed such that it can at least be switched on and off.

37. The device as claimed in claim 21, further comprising an evaluation unit for determining at least one of an activity and a rate of the metabolism.

38.-40. (canceled)

41. The device as claimed in claim 21, further comprising at least one of an alphanumeric and graphic display unit.

42. The device as claimed in claim 41, further comprising an alarm transmitter unit.

43. The device as claimed in claim 42, further comprising an input unit for entering data.

44. The device as claimed in claim 43, further comprising a dosing unit for at least one medicament.

45. The device as claimed in claim 44, further comprising an injection unit for the contrast agent.

46. The device as claimed in claim 45, further comprising an interface unit for at least one of wireless and cable-bound transmission of data.

47. The device as claimed in claim 46, further comprising means for enriching the contrast agent.

48. A device for carrying out the method of claim 1, further comprising a detector for detecting a component of the molecular contrast agent that highlights the at least one substance.

49. A device for detecting at least one substance in a metabolic process of a living being, comprising:
a detector for detecting a molecular contrast agent, administered to the living being and able to highlight the at least one substance, continuously over time at least one location of the living being.

50. The device of claim 49, wherein the detector is integrated in the device.

51. The device of claim 49, wherein the detector is a detector for measuring fluorescence signals.

52. The device of claim 50, wherein the detector is a detector for measuring fluorescence signals.

53. The device of claim 50, wherein the detector includes a CCD chip.

54. The device of claim 51, wherein the detector includes a CCD chip.

55. The device of claim 52, wherein the detector includes a CCD chip.

56. The device of claim 49, further comprising:
a memory for storing variations, over time, of a measurement signal recorded by the detector.

57. The device as claimed in claim 49, further comprising:
means for recording results of the continuous detection over time.

58. The device of claim 57, further comprising:
a memory for storing variations, over time, of the recorded results.

59. The device as claimed in claim 49, further comprising at least one of an alphanumeric and graphic display unit.

60. The device as claimed in claim 49, further comprising an alarm transmitter unit.

61. The device as claimed in claim 49, further comprising an input unit for entering data.

62. The device as claimed in claim 49, further comprising a dosing unit for at least one medicament.

63. The device as claimed in claim 49, further comprising an injection unit for the contrast agent.

64. The device as claimed in claim 49, further comprising an interface unit for at least one of wireless and cable-bound transmission of data.

65. The device as claimed in claim 49, further comprising means for enriching the contrast agent.
